(19) United States
(12) Patent Application Publication
(10) Pub. No.: US 2014/0357977 A1
(43) Pub. Date: Dec. 4, 2014

(54) Catheter Placement Detection System and Method for Surgical Procedures

(71) Applicant: Jun Zhou, Troy, MI (US)

(72) Inventor: Jun Zhou, Troy, MI (US)

(73) Assignee: William Beaumont Hospital, Royal Oak, MI (US)

(21) Appl. No.: 14/117,323

(22) PCT Filed: May 9, 2012
(86) PCT No.: PCT/US12/36988

§ 371 Date: Aug. 22, 2014

Related U.S. Application Data
(60) Provisional application No. 61/485,428, filed on May 12, 2011.

(51) Int. Cl. A61N 5/10; A61B 5/06

(52) U.S. Cl. CPC A61N 5/103; A61B 5/065; A61N 5/1007; A61N 2005/1024
USPC 600/409; 600/8

(57) ABSTRACT

In order to increase the accuracy and speed of catheter reconstruction in surgical procedures such as an HDR prostate implant procedure, an automatic tracking system is provided preferably using an electromagnetic tracking device. The system uses a transmitter with a sensor used for catheter position. Due to substantial interference in the electromagnetic field from the surgical table, implant stepper/stabilizer etc, a calibration algorithm using a scattered data interpolation scheme is implemented to correct tracking location errors. The invention includes methods and systems used to carry out the methods.

### FIELD OF THE INVENTION

This invention relates to methods and systems usable in human and animal surgical procedures. For example, the invention is applicable in the field of human brachytherapy treatment procedures.

### BACKGROUND OF THE INVENTION

In typical brachytherapy surgical procedures, a physician inserts a number of hollow catheters into a target structure within the human body. The number and location of the catheters is determined by a treatment plan, prescribed by a physician based on imaging studies usually done prior to treatment and many other factors. Often, a grid-like guide template structure is used as a guide for catheter insertion having insertion passages arranged in an orthogonal grid pattern. After inserting a number of such catheters at the prescribed loading position and depth, radioisotope sources are either placed permanently in the tissue as “seeds” (low dose rate or LDR brachytherapy), or are loaded into the catheters and are moved robotically inside the catheter to expose tissue surrounding the catheter to a desired radiation dose and then removed (high dose rate “HDR” brachytherapy). The radiation exposure dose is intended to cause radiotoxicity and destroy targeted human tissue, for example cancerous tumors or other structures. One application of this technique is in the area of human prostate brachytherapy. Among other applications, these techniques are also useful for human esophageal brachytherapy.

In human prostrate brachytherapy, many catheters are placed at desired positions using a locating template, positioned on the patient's perineum. However, due to structural characteristics of the catheters, their tips, and density variations in the human tissue, the insertion paths and final positions of the catheters cannot be assumed to be along straight lines extending from the template. Since the actual position of the catheters is critical to provide desired dose application, the radiologist needs confirmation of the catheter placements. This is presently done through ultrasonic imaging procedures. Unfortunately, the ultrasonic procedure used for human prostrate brachytherapy does not provide a clear image of catheter placement. There are numerous artifacts in the image reconstruction and, moreover, there are fundamental limits in the use of a rectally inserted ultrasonic probe during catheter placement procedures. For a real-time ultrasound guided HDR prostate implant procedure, catheter reconstruction has always been challenging and time consuming. This is due in part to many factors including high speckle noise, inter-needle interference, artifacts from calcifications, hyper-echoic tissues, and coil markers for external beam treatment. Furthermore, the catheters are always not straight. They are often curved either inadvertently, or intentionally to reduce normal tissue dose and increase conformity, making the reconstruction of catheter geometry even more difficult.

In view of the foregoing, there is a need for a detection system which provides higher accuracy and a reduction in evaluation time for verifying catheter placement for procedures such as LDR or HDR brachytherapy.

This invention describes a novel system to perform real-time catheter tracking. This system will significantly improve catheter reconstruction speed and accuracy while increasing operator confidence in precise dose delivery.

### BRIEF DESCRIPTION OF THE DRAWINGS

FIG. 1(a) is a schematic diagram of an electromagnetic tracking system in accordance with one embodiment of the present invention.

FIG. 1(b) is a pictorial view of an electromagnetic tracking system in accordance with one embodiment of the present invention.

FIG. 2 is a screenshot of a graphical user interface (GUI) in accordance with an embodiment of the present invention.

FIGS. 3(a)-3(f) are graphical views of catheter tracking results produced by an embodiment of the present invention before calibration; FIGS. 3(a), 3(c), and 3(e), and after calibration; FIGS. 3(b), 3(d), and 3(f). FIGS. 3(a) and 3(b) are x-y plots, FIGS. 3(c) and 3(d) are x-z plots, and FIGS. 3(e) and 3(f) are y-z plots.

FIG. 4(a) is a graphical view of tracking results of catheter placement produced by an embodiment of the present invention.

FIG. 4(b) is a graphical view of tracking results of catheter placement produced using CT-based catheter reconstruction.

### DETAILED DESCRIPTION OF THE INVENTION

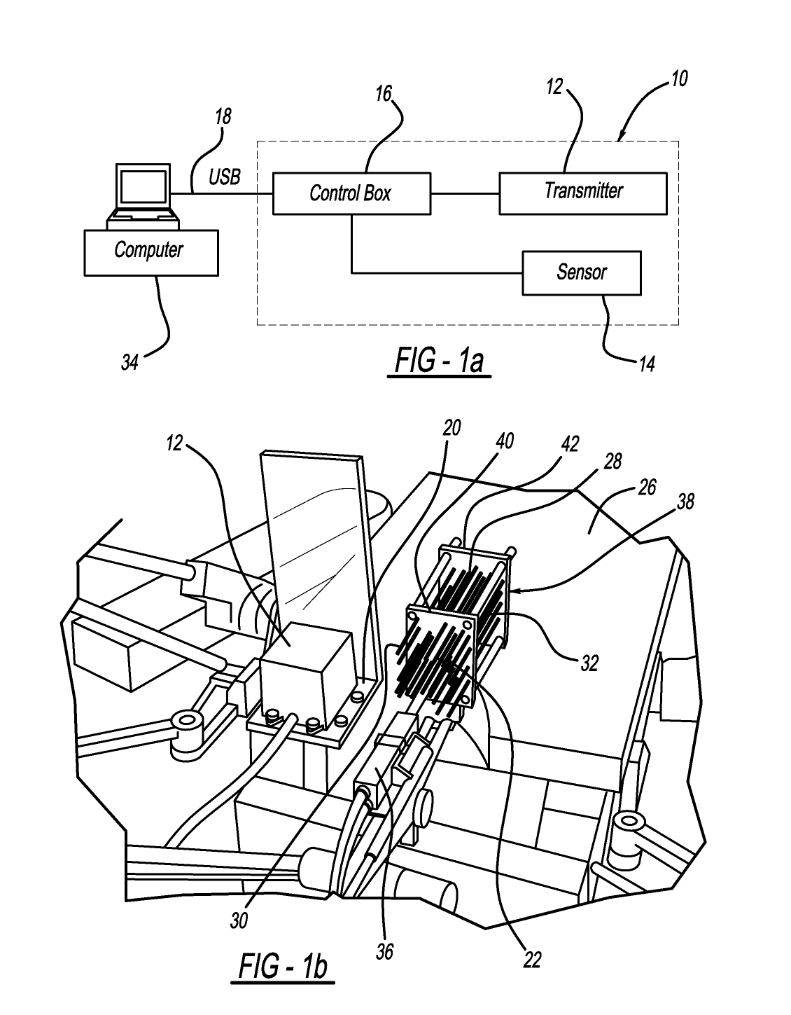

In accordance with this invention, an electromagnetic tracking system 10 is employed. The tracking system 10 as shown in FIG. 1(a) utilizes a transmitter unit 12, preferably one using so-called passive magnetic DC technology (e.g. products available from Ascension Technology Corporation including their “3D Guidance driveBAY”, or “3D Guidance trakSTAR” systems). It is also possible to other tracking systems 10 in accordance with this invention, including those using passive magnetic AC technology. Tracking system 10 include the transmitter 12 mentioned previously, along with one or more miniature sensors 14 which are small enough in size to be inserted into brachytherapy catheters 22 (catheters 22 may also be referred to as “needles”), shown in FIG. 1(b). The system 10 allows the relative position between the transmitter 12 and sensor 14 to be detected and displayed. Catheters 22 have a distal end 28, proximal end 30, and a hollow lumen 32 therebetween.

Systems utilizing passive magnetic DC (or AC) technology like system 10 are inherently influenced by surrounding structures of magnetic materials. In the particular applications considered here, a patient on a surgical couch or operating table 26 during a brachytherapy catheter placement procedure has numerous metallic structures near the surgical site, including the table, surgical tools, and the brachytherapy catheter placement system. These metallic structures are sources of interference. It is therefore necessary in accordance with this invention to correct measured position values using the aforementioned passive magnetic DC (or AC) technology systems to actual positions. For other electromagnetic systems for example using radio frequency or other location systems, it is expected that structures of the surgical site will also be sources of measurement interference requiring correction, thereby also requiring correction.

Both the transmitter 12 and the sensor 14 are connected to control box 16 controlled by a computer 34 through USB cable 18. An exemplary transmitter 12 has a range of 36 cm and is placed on a supporting bracket 20, as shown in FIG. 1(b), that can be positioned close to the surgical site and the catheters 22. An exemplary sensor 14 has a diameter of 0.9 mm and can be inserted into 16-gauge needles or catheter lumens 32. FIG. 1(b) further shows an ultrasonic probe attached to a stepper unit to move forward and backward for imaging the prostate as part of HDR brachytherapy treatment. That figure further shows a three-dimensional grid like phantom structure 38 used to demonstrate the present invention, and provide system calibration. Structure 38 has grid plates 40 and 42 having apertures for receiving catheters 22 and positioning them in desired orientations.

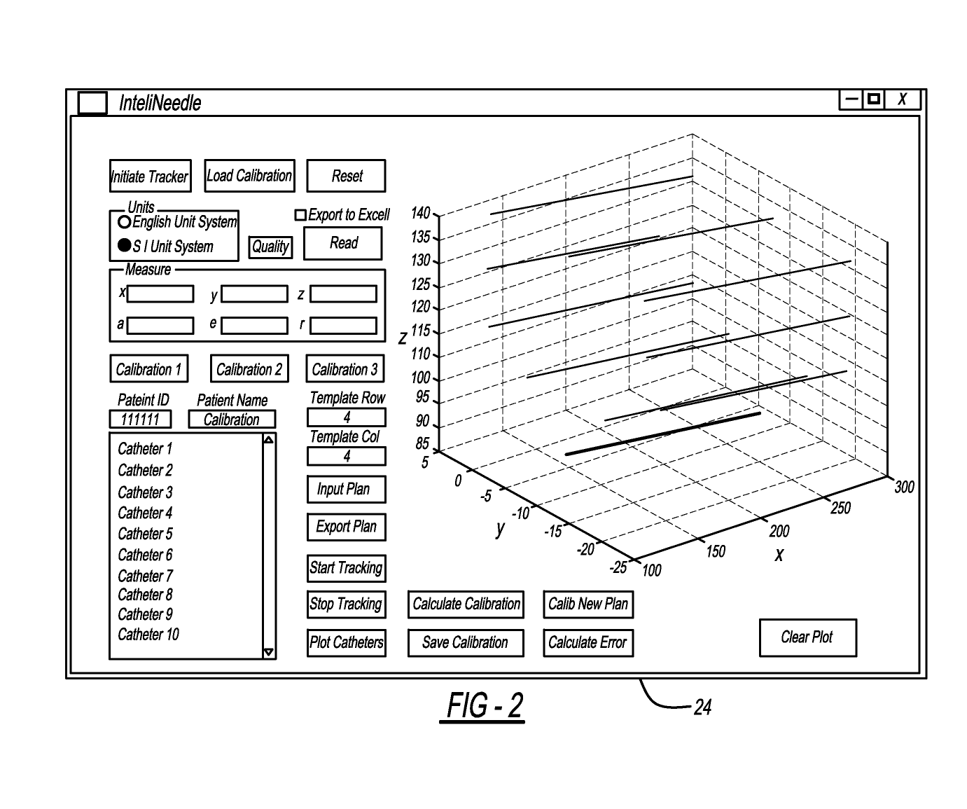

FIG. 2 shows the graphical user interface (GUI) image 24 of the program used to control the system 10. The tracking process in accordance with this invention is conducted in the following steps: 1) after finishing insertion of a plurality of catheters 22 into the patient at the surgical site, sensor 14 is inserted into the proximal end 30 of one catheter 22, and driven to the distal end 28; 2) click the “Start Tracking” button on the GUI and then retract the sensor 14 out of the catheter 22; 3) once the sensor 14 is out of the catheter 22, click the “Stop Tracking” button on the GUI. During the above process, transmitter 12 and sensor 14 are activated to provide tracking. The tracking data corresponds to the catheter 22 will be saved to the plan; 4) go to the next catheter 22 and repeat the previous steps for all catheters; 5) apply calibration (described below) to the tracking result (the calibration can also be applied during the tracking process); 6) export the tracking results (RT plan) to the treatment planning system for planning. Since the sensor 14 is physically constrained to move along the catheter lumen 32, detecting its path also describes the shape and position of the inserted catheters 22. Calibration could also be conducted during insertion of sensor 14, i.e. “Start Tracking” could be done during sensor 14 insertion rather than during retraction as mentioned above. Moreover, tracking could be done in both directions if desired.

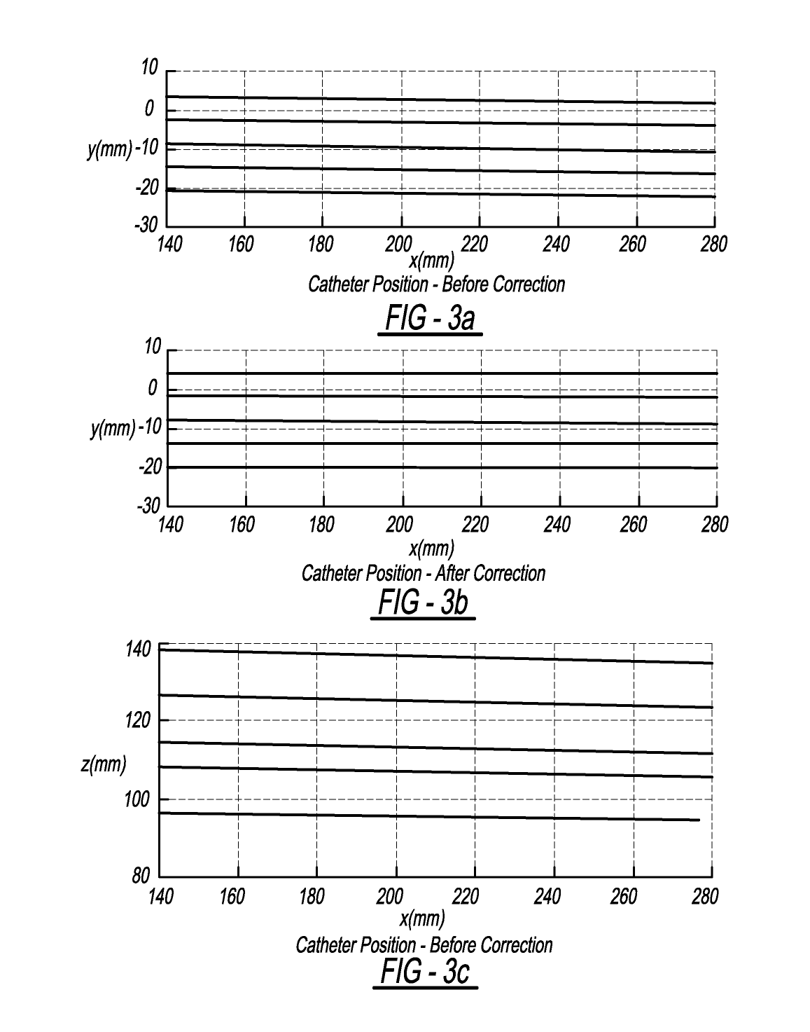

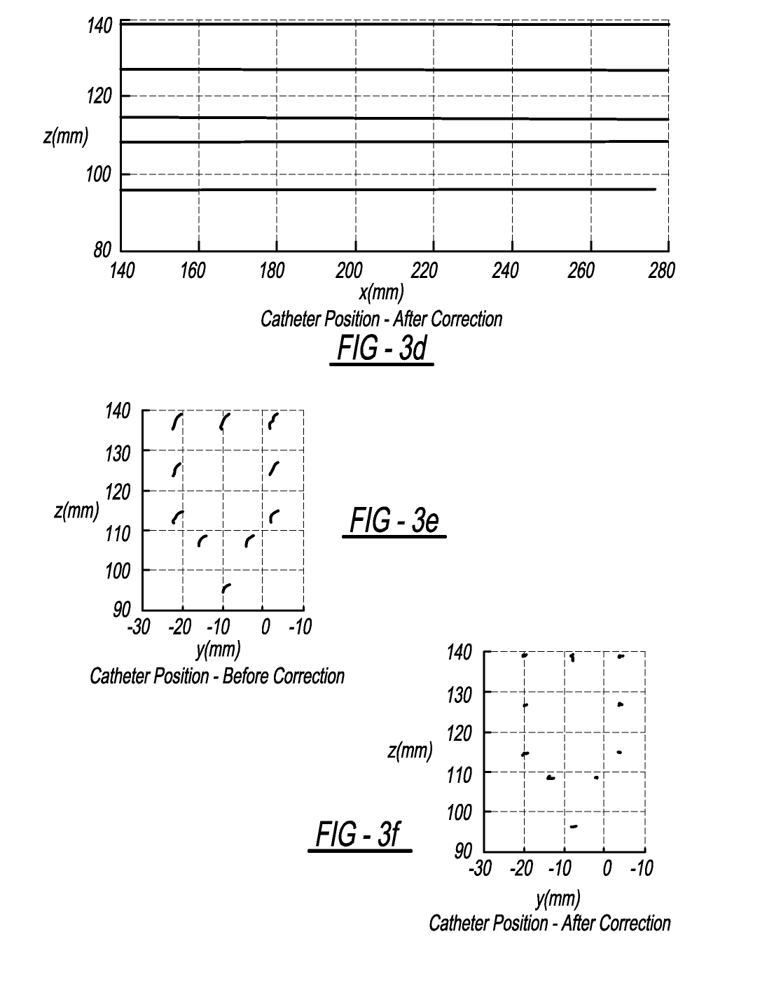

Calibration is accomplished using a calibration algorithm involving a scattered data interpolation scheme. The QA phantom structure 38 with known catheter positions (shown in FIG. 1(b)) is used for calculating calibration profiles. FIGS. 3(a)-3(f) shows orthogonal views of the tracking results for the 10 catheters 22 displayed in the right panel of FIG. 2 using phantom 38. The reconstruction results before correction (FIGS. 3(a), 3(c), and 3(e)) and after correction (FIGS. 3(b), 3(d), and 3(f)) are shown. As shown in FIGS. 3(a), 3(c), and 3(e), the system's accuracy degrades as the sensor-transmitter distance increases. In one experiment using the present invention tracking at distances of 140 mm to 280 mm was conducted. However, after calibration, the error can be minimized as shown in FIGS. 3(b), 3(d), and 3(f). Once the actual positions of the catheters 22 are known, treatment plan modification can be made to provide desired dosing. Once the calibration factors for a particular surgical arrangement are developed using the phantom structure 38, the assumption is made that patient-to-patient differences are small as related to the calibration. The calibration factors determined as described above are used to modify detected positions of catheters positioned in a patient to more closely determine actual catheter placement.

As mentioned previously, calibration is needed due to the influences of surrounding magnetic structures and other sources of interference. Even without such interference however, calibration will be needed since outputs are affected by the position of transmitter 12 relative to catheters 22. Accordingly, it is necessary that the relationship between the position of transmitter 12 and the catheters 22 is reproduced between establishing the correction process using the phantom structure 38 and during surgical procedures.

As a reproducibility study for the present invention, the calibration profiles were tested under various equipment arrangements. While the profiles are sensitive to the relative position between the transmitter 12 and the operating table 26, reasonable position variations of the stepper, ultrasound machine, and leg stirrups (sources of transmitter-sensor tracking errors) introduce <1 mm error.

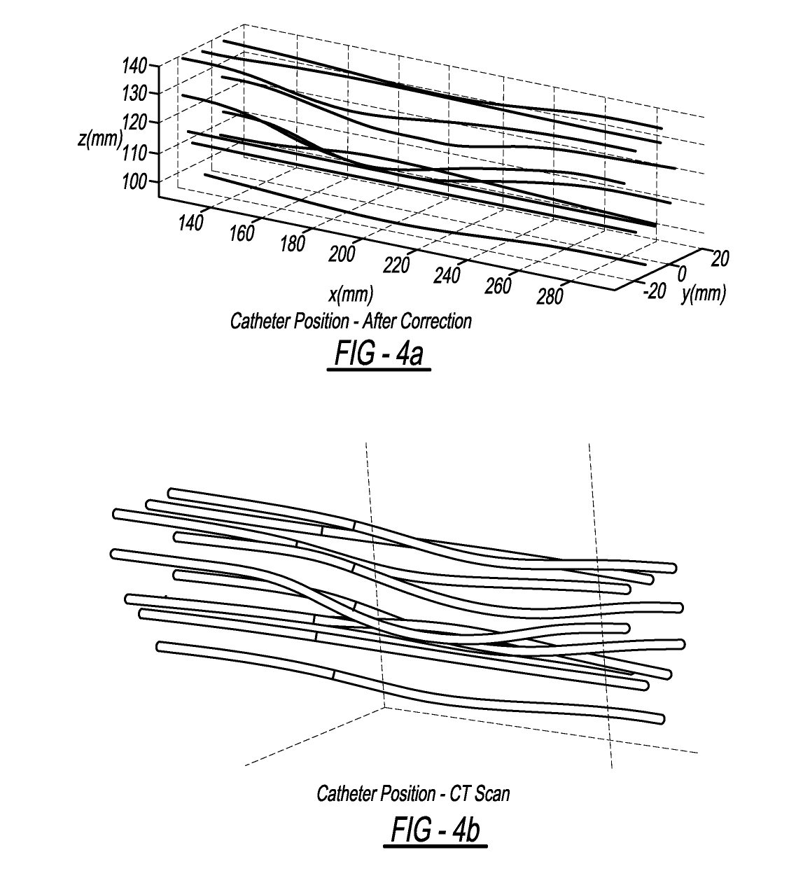

To further validate the system 10, straight catheters 22 in the QA phantom structure 38 were bended and tracked with the system as shown in FIG. 4(a). To verify the corrected catheter positions, the phantom 38 was then scanned with CT (computed tomography) and the catheters 22 were reconstructed in the Oncentra® Brachy, as shown in FIG. 4(b). The CT scanned positions are used as a baseline of actual catheter positions. It should be noted that CT scanning of catheter placements is not preferred for patient use due to cost, complexity, and patient radiation dose exposure, but is used here to validate the inventive approach. In an experiment for demonstrating the present invention, average tracking accuracies after calibration were found to be 0.4±0.3 mm; and 2.4±1.7 mm without calibration. The max standard deviation was 0.9 mm in the test range for the reproducibility test. Thus, the calibration steps used in this invention significantly improved catheter position determination. The total tracking time for ten catheters 22 was less than four minutes and the reconstruction result matches CT data within 2.0 mm.

Compared to conventional ultrasound based real-time catheter reconstruction method in the HDR prostate implant; the system 10 of this invention can reduce the error from >3 mm to <1.5 mm, and shorten the procedure time from 15-60 minutes to <4 minutes. Furthermore, this technique can also be used for other HDR implants.

While the present invention has been described in terms of certain preferred embodiments, it will be understood that the invention is not limited to the disclosed embodiments, as those having skill in the art may make various modifications without departing from the scope of the following claims.

What is claimed is:

1. A method for reconstructing a catheter path in a surgical procedure, the method comprising:
inserting at least one catheter into a target structure of a patient's body, the at least one catheter having a distal end disposed inside the patient's body, a proximal end disposed outside the patient's body, and a catheter lumen extending between the proximal and the distal ends;
inserting a sensor into the catheter lumen through the proximal end of the at least one catheter;
moving the sensor through the catheter lumen; and
tracking the position of the sensor while moving the sensor through the catheter lumen to determine the path.

2. The method of claim 1 wherein the step of tracking the position comprises using a sensor and transmitter using passive magnetic DC or AC operation.

3. The method of claim 1, wherein the inserting a sensor step further comprises wherein the moving the sensor step comprises retracting the sensor longitudinally through the catheter lumen from the distal end to the proximal end of the catheter or inserting the sensor into the proximal end toward the distal end.

4. The method of claim 1, wherein the tracking the position of the sensor step comprises:
acquiring sensor tracking data while moving the sensor through the catheter lumen of a plurality of the catheters; and
applying a set of predetermined calibration correction factors to the sensor tracking data to determine the paths of the plurality of catheters.

5. The method of claim 4, further comprising the steps of providing a phantom structure providing known positions for the catheter, tracking the path of the sensor in the plurality of the catheters, and determining the correction factors by comparing the tracked sensor paths to the known positions.

6. The method of claim 4, wherein the surgical procedure is a brachytherapy procedure.

7. The method of claim 6, wherein the target structure of the patient's body is the patient's prostate.

8. The method of claim 6, wherein the method further comprises the step of inserting a radioactive source into the at least one catheter as part of a HDR brachytherapy procedure and further comprising the step of applying a treatment plan based on the paths of the plurality of catheters.

9. The method of claim 8, wherein the target structure of the patient's body is the prostate gland.

10. A method for reconstructing a catheter path in a surgical procedure, the method comprising:
providing a phantom structure providing known positions for a plurality of catheters, the catheters each having a distal end, a proximal end, and a catheter lumen formed between the proximal end and the distal end,
tracking the path of a sensor inserted into the plurality of catheters positioned in the phantom structure,
determining position correction factors by comparing the tracked paths of the sensor in the catheters in the phantom structure to the known positions provided by the phantom structure,
inserting the catheter into a target structure of a patient's body,
inserting the sensor into the catheter lumen through the proximal end of one of the catheter in the patient's body;
moving the sensor longitudinally through the catheter lumen in the patient's body;
tracking the sensor while moving the sensor through the catheter lumen in the patient's body to determine the catheter path, and
applying the position correction factors to determine actual positions of the catheter in the patient's body.

11. The method of claim 10 wherein the step of tracking the position comprises using a sensor and transmitter using passive magnetic DC or AC operation.

12. The method of claim 10, wherein the method further comprises the step of inserting a radioactive source into the at least one catheter in the patient's body as part of a HDR brachytherapy procedure and further comprising the step of applying a treatment plan based on the paths of the plurality of catheters.

13. The method of claim 12, wherein the target structure of the patient's body is the patient's prostate.

14. A system for reconstructing a path of a catheter in a surgical procedure, the system comprising:
a sensor configured to be inserted into and to be moved through the catheter;
a transmitter configured to send signals to the sensor as the sensor is moved through the catheter;
a control box in communication with the sensor and the transmitter, the control box being configured to acquire sensor tracking data from the transmitter or the sensor as the sensor is moved through the catheter; and
a computer in communication with the control box, the computer being configured to receive the sensor tracking data from the control box and to determine the path of the catheter by applying a set of predetermined calibration correction factors to the sensor tracking data.

15. The system in accordance with claim 14 further comprising the sensor and transmitter operating using passive magnetic DC or AC technology.

16. The system in accordance with claim 14 further comprising a phantom structure for positioning a plurality of the catheters in a predetermined orientation for enabling a calibration of the positions of the catheters and for creating the correction factors.

17. The system in accordance with claim 14 further comprising a bracket for positioning the transmitter in a predetermined orientation with respect to the catheters.

18. A system in accordance with claim 14 wherein the surgical procedure is HDR brachytherapy and further comprising the computer applying a treatment plan based on the paths of the plurality of catheters.

19. A system in accordance with claim 18 wherein the surgical procedure is HDR brachytherapy of the human prostate gland.
